(19) United States
(12) Patent Application Publication
(10) Pub. No.: US 2011/0143304 A1
(43) Pub. Date: Jun. 16, 2011

(54) Adaptor for Lighted Dental Device

(75) Inventors: Mohammad A. Jamnia, Chicago, IL (US); James Feine, Bellaire, TX (US)

(73) Assignee: HU-FRIEDY MFG. CO., INC., Chicago, IL (US)

(21) Appl. No.: 12/635,736

(22) Filed: Dec. 11, 2009

(51) Int. Cl. A61B 1/24; F21V 33/00; H02J 7/00

(52) U.S. Cl. 433/29; 362/253; 320/108

(57) ABSTRACT

Embodiments of an adaptor for a lighted magnetostrictive device, a lighting system for a hand-held dental delivery device including an adaptor, and a method of providing light at a magnetostrictive hand-held device using an adaptor are disclosed. The adaptor may securely connect to a hand-piece of the device, and may include a self-contained electric power storage device to power a light source, an adaptor light channel for delivering light from a light source to the insert, or both. The light source may be disposed within the hand-piece, the adaptor or the insert. A user may switch the light source on and off, and/or may dim or brighten an intensity of the light. The light source may provide illumination even when a tip of the device is not activated, and the electric power storage device may be inductively re-charged.

### BACKGROUND OF THE DISCLOSURE

1. Field of the Disclosure

This disclosure relates generally to hand-held dental devices, and in particular, to providing light for hand-held magnetostrictive dental devices by using an adaptor.

2. Background Description

Magnetostrictive ultrasonic dental devices are used in dentistry to remove calculus from teeth and perform other cleaning or abrasive operations by vibrating a metal insert at an ultrasonic frequency. A magnetostrictive ultrasonic dental device typically receives electric current having a controlled frequency from a generator and translates the received electrical energy into a mechanical motion of a tip of an insert coupled to a hand-piece. To this end, a magnetostrictive dental device hand-piece includes an electrical connector, a coil, and a housing functioning as a handle. Alternating current provided to the coil induces a corresponding alternating magnetic field. At a resonant frequency, a stack of metal plates disposed within the coil may vibrate in response to the alternating magnetic field, and these vibrations may be transferred to the tip.

Since a mouth is a small and dimly-lit space in which to work, it is desirable to have an ultrasonic dental tool that can bring light directly into and around the working area, e.g., tooth and gumline surfaces. It is further desirable to omit an additional power cord for the light to minimize the number of obstructions that may get in the way of a dental practitioner's task at hand. Several known approaches exist where light is delivered to the tip end of a magnetostrictive insert by using available power in the coil of the hand-piece without needing an additional power cord.

In one known approach, a magnetostrictive ultrasonic dental insert includes a first coil or transducer for generating ultrasonic vibrations and producing the mechanical motion of the dental tip. A second coil or transducer generates a voltage signal in response to the mechanical movement. A light source in the vicinity of the tip receives the voltage signal from the second transducer and illuminates. This approach has several disadvantages. First, the intensity of the generated light may vary based on power fluctuations delivered to the first coil, such as when an operator varies tip vibration rate by varying the power to the hand-piece. Second, the intensity of the light may vary based on the integrity of the stack itself. Furthermore, this approach does not allow the light source to be turned on when the tip is not vibrating, as the voltage generated by the second transducer and used to illuminate the light source necessarily requires energizing the first transducer and stack.

In another known approach, a magnetostrictive ultrasonic dental device includes a similar primary coil for generating ultrasonic vibrations and the mechanical motion of the dental tip. A secondary coil is positioned to be inductively coupled to the primary coil and electrically connected to the light source. The secondary coil is oriented so that a magnetic field induced by energizing the first coil induces, in turn, a current flow in the second coil that causes the light source to illuminate. Like the first discussed approach, this other approach also suffers from varying intensities of the light source based on power fluctuations delivered to the first coil. Similarly, in this approach, the light source may not be turned on independent from tip vibration.

In yet another known approach, light may be piped from a light source in an ultrasonic generator to the ultrasonic dental device. At the ultrasonic dental device, the light may be delivered through hand-piece tubing, the hand-piece, and the insert to the tip. This approach, however, requires the ultrasonic dental device to be used with a generator equipped with a light source.

### BRIEF SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Embodiments of an adaptor for use with a lighted magnetostrictive device are disclosed. In some embodiments, the adaptor may include a housing and a self-contained electrical storage device disposed within the housing. (The terms “electrical storage device,” “electric power storage device,” and “power pack” are interchangeably used herein, and are described in more detail in a later section.) The self-contained electrical storage device may power a light source to illuminate an area proximate to the lighted magnetostrictive device. The adaptor may be removably attached to a hand-piece of the lighted magnetostrictive device, and the light source may be disposed within the adaptor, the hand-piece or the insert.

In other embodiments, the adaptor may include a housing and an adaptor light channel disposed therein and configured to deliver or channel light from a light source to an insert of the lighted magnetostrictive device, so that the light may be channeled from the adaptor, through the insert, and to an area external and proximate to the lighted magnetostrictive device. The adaptor may be removably attached to the hand-piece of the lighted magnetostrictive device. The light source may be disposed within the adaptor or the hand-piece, and may be powered by an electric power source that provides electric power independently of a magnetic field generated by the lighted magnetostrictive device.

Embodiments of a lighting system for a hand-held dental delivery device are disclosed. In some embodiments, the lighting system may include a hand-piece of the hand-held dental delivery device and an insert coupled to the hand-piece, where the insert includes a tip and an insert light channel configured to channel or deliver light through the insert to illuminate an area proximate to the tip. The lighting system may include an adaptor that is removably coupled to the hand-piece and includes an electrical storage device for powering a light source. The electrical storage device may be self-contained, and may be configured to provide electric power independently of a magnetic field generated by the dental delivery device. The light source powered by the electrical storage device may be disposed in the hand-piece, the adaptor or the insert.

In some embodiments, the lighting system may include the hand-piece of the hand-held dental delivery device, the insert coupled to the hand-piece including the tip, and an insert light channel configured to channel or deliver light through the insert to an area proximate to the tip. The lighting system may include an adaptor that is removably coupled to the hand-piece and includes an adaptor light channel configured to channel or deliver light to the insert. The light source may be disposed in the hand-piece or the adaptor, and may be powered by a source that is configured to provide electric power independently of a magnetic field generated by the dental delivery device.

Embodiments of a method of providing light at a magnetostrictive hand-held device are disclosed. In some embodiments, the method may include engaging an insert within a hand-piece of the magnetostrictive hand-held device; attaching an adaptor to the hand-piece, where the adaptor includes a self-contained electrical storage device for powering a light source; abutting the insert to the adaptor; and disposing the light source in the insert, the adaptor or the hand-piece.

In some embodiments, the method may include engaging an insert within a hand-piece of the magnetostrictive hand-held device; attaching an adaptor to the hand-piece, where the adaptor includes an adaptor light channel for delivering or channeling light from a light source to the insert; and coupling the light source to an electric power source that is configured to provide electric power independently of a magnetic field generated by the dental delivery device.

The embodiments of the present disclosure provide numerous benefits. For example, in some embodiments of the present disclosure, the light source is powered by an electric power storage device or power pack that is different from the power source of the magnetostrictive device. Thus, power fluctuations to the device do not affect the intensity of the light source at all. By using the electrical storage device, the light source generally may deliver a light at a steady, desired level of intensity with minimal fluctuations. Similarly, by using the electrical storage device, power (and therefore, light intensity) fluctuations are divorced from an integrity of a stack within a magnetostrictive device. In some embodiments, the electrical storage device may be conveniently re-charged via induction.

Furthermore, the embodiments of the present disclosure allow the light source to be illuminated even when a tip of a magnetostrictive device is not moving. In fact, the present disclosure provides embodiments that allow a user to control whether the light source is on or off independently, from a user perspective, of whether or not the tip is activated.

Additionally, embodiments of the present disclosure do not limit the magnetostrictive dental device to being used with only generators that are equipped with a light source, but allow the magnetostrictive dental device to be used with any type of generator. The present disclosure also eliminates an extra light pipe between a generator and the dental device to further minimize the number of obstructions that may get in the way of a dental practitioner's task at hand.

### BRIEF DESCRIPTION OF SEVERAL VIEWS OF THE DRAWING

FIG. 1 schematically illustrates an ultrasonic dental system (including a signal generator, a hand-piece, and a dental insert) that may operate in conjunction with the present disclosure;

FIG. 2 is a cutaway view of the dental system of FIG. 1 with the insert engaged within the hand-piece;

FIG. 3A illustrates an external view of an embodiment of a magnetostrictive device with an adaptor securely attached;

FIG. 3B illustrates an external view of the embodiment of FIG. 3A with the adaptor unattached to the magnetostrictive device;

FIG. 4A includes a cutaway view of the magnetostrictive device of FIG. 3A, where the adaptor is securely attached to the hand-piece of the magnetostrictive device;

FIG. 4B is an enlarged, detailed view of a portion of FIG. 4A;

FIG. 5A includes a cutaway view of the magnetostrictive device of FIG. 3A, where an insert is engaged within the hand-piece and the adaptor is securely attached to the hand-piece;

FIG. 5B is an enlarged, detailed view of a portion of FIG. 5A;

FIG. 6A illustrates a horizontal, external view of the magnetostrictive device of FIG. 5A;

FIG. 6B illustrates a different external view of the magnetostrictive device of FIG. 6A;

FIG. 7 is a cutaway view of an embodiment of a lighting system for a hand-held dental device including a hand-piece of a magnetostrictive device, an insert with a tip, and an adaptor with a self-contained electrical power source disposed within its walls;

FIG. 8 depicts an embodiment, in block diagram form, of a magnetostrictive ultrasonic device with an induction charger; and

FIGS. 9 and 10 each show different embodiments of a method for providing light at a magnetostrictive hand-held device.

### DETAILED DESCRIPTION OF THE PREFERRED EMBODIMENTS

Although the following text sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments could be implemented, using either current technology or technology developed after the filing date of the application for this patent, which would still fall within the scope of the claims.

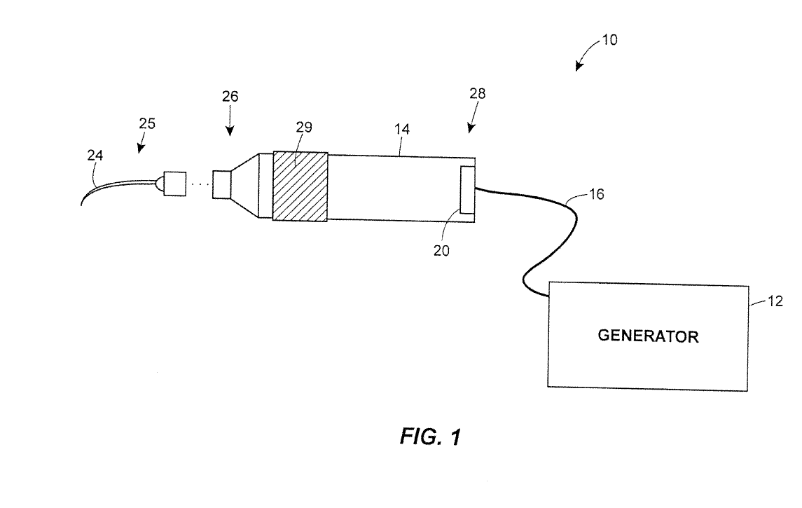

FIG. 1 illustrates an exemplary ultrasonic dental system 10. A dental practitioner may use the dental system 10 to remove calculi from a patient's teeth or to perform scaling, abrasion, or other similar dental procedures. In particular, a generator 12 may generate pulse width modulated (PWM) or alternating electric current (AC) at a controlled, typically ultrasonic frequency such as 25 or 30 KHz. The generator 12 may then supply the current to a hand-piece 14 via a cord 16. The hand-piece 14 may be provided with a port 20 including one or more electrical connectors and, optionally, a fluid connector to receive a supply of liquid, air, or both.

The hand-piece 14 may translate the electrical energy received from the generator 12 into mechanical energy by inducing an electro-magnetic field and applying this field to a vibrating or otherwise movable component. In particular, the hand-piece 14 may vibrate a tip 24 of an insert 25 that is enabled to move in response to the electro-magnetic field. The tip 24 may vibrate at a frequency dependent on the driving AC frequency supplied by the generator 12, the physical dimensions of the tip 24, and the internal circuitry of the hand-piece 14. Although illustrated in FIG. 1 as an integral component, the insert 25 may include several parts, some of which may be further detachable, to facilitate, for example, cleaning, maintenance and recharging. One of ordinary skill in the art will also appreciate that the transducer for converting the energy supplied by the generator 12 into a vibratory motion of the tip 24 may be disposed in the hand-piece 14, in the insert 25, or in a separate component interacting with the hand-piece 14 and the insert 25.

The hand-piece 14 may have a tubular, rectangular, or other elongated form. The insert 25 may engage the hand-piece 14 at a patient-proximal end 26. Accordingly, the port 20 may be disposed at the patient-distal end 28. The operator may hold the hand-piece 14 at or about the grip section 29. Although the grip section 29 may be integral with the body of the hand-piece 14, FIG. 1 schematically illustrates a removable grip made of a different material than the external layer of the hand-piece 14. At least the middle section of the hand-piece 14 may be ergonomically shaped to provide an easy grip for the operator.

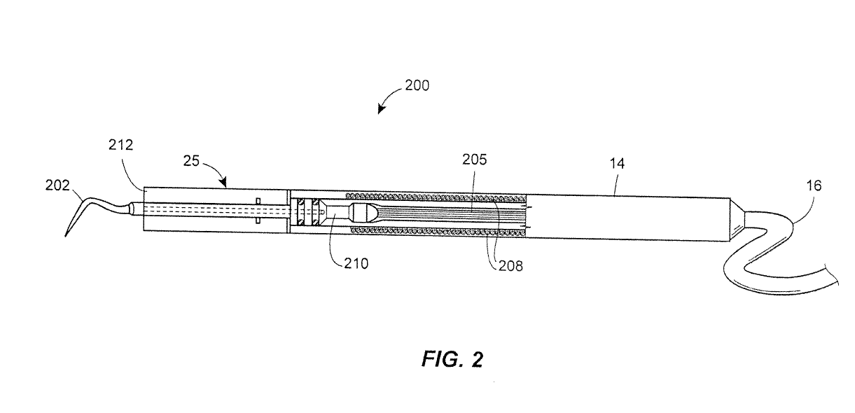

FIG. 2 is a cutaway view 200 of the exemplary dental system 10 with the insert 25 insertably engaged with the hand-piece 14. The hand-piece 14 may be connected with the cord 16 that may provide the electrical connection to the generator to drive a tip 202 of the magnetostrictive insert 25.

The insert 25 may include a magnetostrictive stack 205 typically formed of nickel or nickel alloy plates that may be soldered together at each end. When the insert 25 is insertably engaged with the hand-piece 14, the stack may be encircled by, or may be in proximity to, a coil 208 in the hand-piece 14. The coil 208 may receive electrical energy via the cord 16, and may induce a magnetic field. The stack 205 may respond to the induced magnetic field. For example, at a corresponding resonant frequency, the stack 205 may vibrate in response to the magnetic field. Although FIG. 2 illustrates a magnetostrictive stack 205 of thin metal plates, in other embodiments, any type, shape and/or configuration of a magnetic element (e.g., the stack, one or more rods, a pin, etc.) responsive to a magnetic field may be used. The magnetic element may be formed, at least partially, from any suitably magnetic, ferromagnetic, diamagnetic, and/or paramagnetic material that exhibits or can be made to exhibit attraction and/or repulsion to a magnetic or electromagnetic field.

The stack 205, the one or more rods or other magnetic element may be connected to an ultrasonic horn or a velocity transducer 210 which may, in turn, be connected to the tip 202 covered with a grip 212. In this illustration, the tip 202 is illustrated as a scaling tip, but any known tip for a dental procedure may be used in conjunction with embodiments of the present invention. The velocity transducer 210 may be integral with the tip 202 or may be threaded to provide a threaded connection with the tip 202. Thus, a vibration of the stack 205 in response to an induced magnetic field at a resonant frequency may cause (via the velocity transducer 210) the tip 202 to vibrate accordingly. As known in the art, the resonant frequency may not be an exact, precise frequency. Real-world factors such as impedance and a physical condition of the stack or magnetic element may come into play, so that a resonant frequency may include a narrow range of frequencies around an exact frequency that may still effect the vibration of the stack 205.

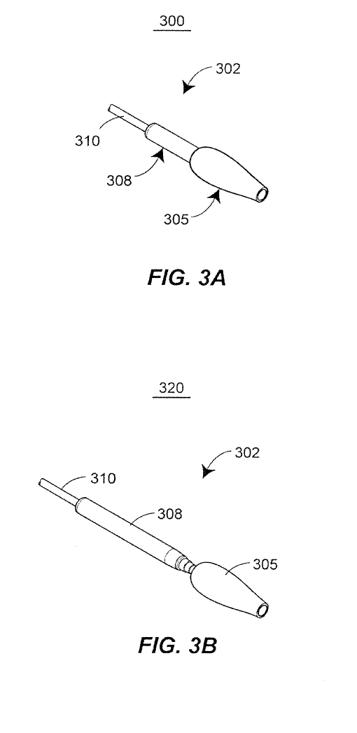

FIG. 3A illustrates an external view 300 of an embodiment of a magnetostrictive device 302 with an adaptor 305. The magnetostrictive device illustrated in FIG. 3A may be (but is not required to be) a hand-held dental device and may operate in conjunction with the ultrasonic dental system 10 of FIGS. 1 and 2. The external view 300 illustrates the adaptor 305 securely connected to a hand-piece 308 of the device. The adaptor 305 may have a generally annular shape into which the hand-piece 308 may be inserted. A cord 310 connects the hand-piece 308 to an ultrasonic generator (not shown).

FIG. 3B illustrates an external view 320 of the magnetostrictive device 302 of FIG. 3A with the adaptor 305 removed from the hand-piece 308. In the embodiments illustrated in FIGS. 3A and 3B, the adaptor 305 connects to the hand-piece 308 by sliding around the hand-piece 308 into a secure position. The adaptor 305 includes an orifice (not visible) into which the hand-piece 308 is inserted, and may include a securing mechanism (not shown) so that the adaptor 305 may be secured in a desired location along the length of the hand-piece 308. Of course, FIGS. 3A and 3B only illustrate one possible embodiment of connecting an adaptor to a hand-piece 308 of a magnetostrictive device. An adaptor as disclosed herein is not limited to being generally cylindrical or annular, as shown in reference 305 of FIGS. 3A and 3B, but may be of any size or shape. Similarly, an adaptor may not be required to connect to the hand-piece by sliding over the hand-piece, but instead may be attached to the hand-piece using any suitable mechanism or method, such as attaching via a latching mechanism, securely engaging a protrusion of the adaptor into a receptacle of the hand-piece, or engaging in a bayonet fitting.

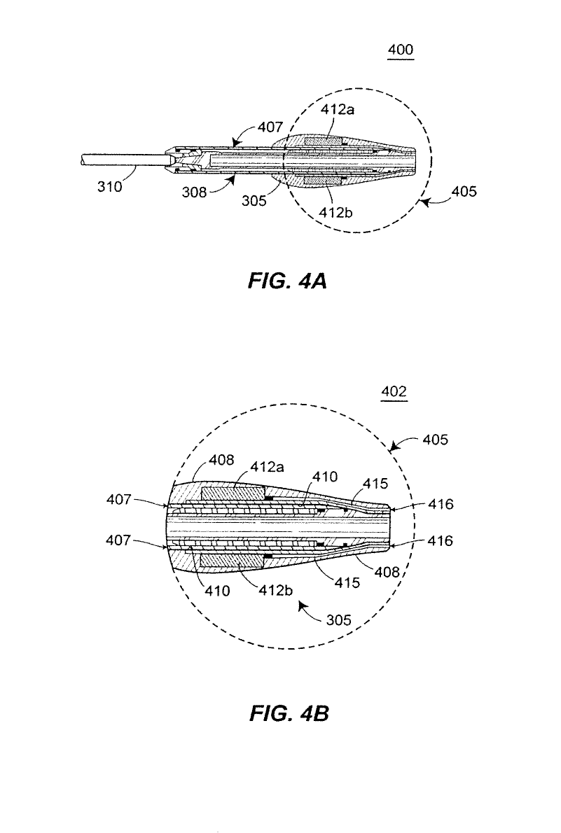

FIG. 4A illustrates a cut-away view 400 of the magnetostrictive device of FIGS. 3A and 3B. FIG. 4B is a enlarged, detailed cut-away view 402 of an area bounded by the circle 405 of the cut-away view 400 of FIG. 4A. In both FIGS. 4A and 4B, the adaptor 305 is securely connected with the hand-piece 308. For purposes of clarity, FIGS. 4A and 4B are concurrently discussed.

As shown in FIGS. 4A and 4B, the adaptor 305 may encircle a housing 407 of the hand-piece 308. The adaptor 305 may include an outer wall 408 and an inner wall 410 between which one or more light sources 412a, 412b may be disposed. While the light sources 412a, 412b are depicted in FIGS. 4A and 4B as a set of light emitting diodes (LEDs), this embodiment is only exemplary. In fact, the light sources 412a, 412b may any known type of light generating source, such as incandescent bulbs, neon bulbs, or light rings. The adaptor 305 may include one or more adaptor light channels 415 that are in connection with the light source(s) 412a, 412b and configured to channel or deliver light through the adaptor and an adaptor light-exiting aperture 416 into an insert coupled to the magnetostrictive device. The one or more adaptor light channels 415 may be one or more light pipes disposed longitudinally along one or more sides of the adaptor 305, or the one or more adaptor light channels 415 may encircle the inner wall 410 of the adaptor 305 along its length. Other configurations of the one or more adaptor light channels 415 may be possible.

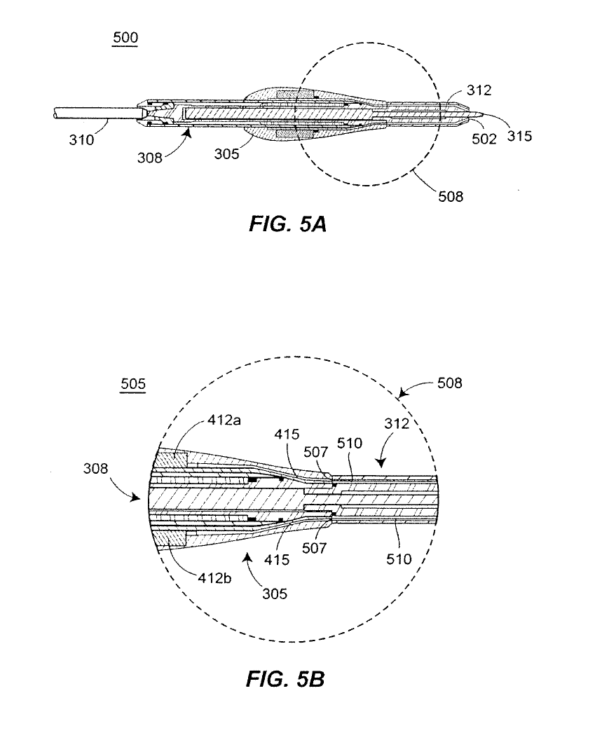

FIG. 5A illustrates a cut-away view 500 of the magnetostrictive device of FIG. 3A with the adaptor 305 securely connected to the hand-piece 308 and an insert 312 with a tip 315 engaged within the hand-piece 308. In the embodiment shown in FIG. 5A, an insert light-exiting aperture 502 is configured and positioned to deliver light outwardly from the insert 312 and to illuminate an area proximate to the tip 315.

FIG. 5B is a enlarged, detailed cut-away view 505 of an area bounded by the circle 508 of FIG. 5A. In just one scenario, FIGS. 5A and 5B may illustrate an example of a lighted, magnetostrictive dental device including an engaged insert 312 and a secured adaptor 305. For purposes of clarity, FIGS. 5A and 5B are concurrently discussed.

When the insert 312 is engaged within the hand-piece 308 and the adaptor 305 is securely connected to the hand-piece 308, at least a portion of the insert 312 may abut at least a portion of the adaptor 305. This abutment may allow the adaptor light channel 415 to be aligned with one or more insert light-entering apertures 507 in connection with one or more insert light channels 510 within the insert 312. The insert light channels 510 may configured to channel or deliver light from the insert light-entering aperture(s) 507, and through the insert 312 to exit the insert at the light-exiting aperture 502. In some embodiments, the insert light-exiting aperture 502 may be proximate to the tip 315 so that the light illuminates an area proximate to the tip 315, but other embodiments may be possible. The one or more insert light channels 510 may be one or more light pipes disposed longitudinally along one or more sides of the insert 312, or the one or more insert light channels 510 may encircle a stack or coil of the insert 312 along its length. Other configurations of the one or more insert light channels 510 may be possible.

In some embodiments, the one or more light source(s) 412a, 412b disposed within the adaptor 305 may be in electrical connection with and powered by a self-contained electrical storage device (not shown) co-disposed within the adaptor 305. The self-contained electrical storage device may generate electric power independent of a magnetic field produced by the magnetostrictive device. The self-contained electrical storage device may be, for example, a single battery, a battery pack, or any suitable type of known disposable, replaceable or rechargeable battery. In some embodiments, the self-contained electrical storage device may be another type of power density device, such as a supercapacitor, an electrochemical double layer capacitor (EDLC), or an ultracapacitor. In fact, any known self-contained electrical storage device may be used in conjunction with embodiments of the present disclosure.

In some embodiments, the one or more light source(s) 412a, 412b disposed within the adaptor 305 may be in electrical connection with and powered by an external electric power source that is externally disposed to the hand-piece 308, the insert 312 and the adaptor 305. For example, the one or more light source(s) 412a, 412b may be coupled to a standard electrical wall outlet. In another example, the one or more light source(s) 412a, 412b may receive electric power from a same electric power source of the magnetostrictive device, such as the generator 12 shown in FIG. 1.

Irrespective of the electric power source by which the one or more light source(s) 412a, 412b receives electrical energy, a user-activated switch (not shown) may be provided to interrupt and re-establish the electrical connection between the electric power source and the one or more light source(s). Thus, via the user-activated switch, a user of the magnetostrictive device may control whether or not the light source is illuminated.

Although the one or more light sources 412a, 412b are illustrated in FIGS. 4A, 4B, 5A and 5B as being disposed within the adaptor 305, this is merely exemplary. In some embodiments, the one or more light sources 412a, 412b may be disposed within the hand-piece 308. In these embodiments, when the insert 312 is engaged within the hand-piece 308 and the adaptor 305 is securely connected to the hand-piece 308, light may be channeled or delivered from the light source in the hand-piece 308 (for example, via a hand-piece light channel) into the adaptor light channel 415, and through the adaptor light channel 415 to exit at the adaptor light-exiting aperture 416 into the insert light-entering aperture(s) 507 and the insert light channel 510. The insert light channel 510 may deliver or channel the light through the insert 312 to the insert light-exiting aperture 502. In these embodiments, the light sources 412a, 412b disposed within the hand-piece 308 may receive electric power from a battery or other self-contained electrical storage device, from the generator connected to the hand-piece, or from a different, external electric power source.

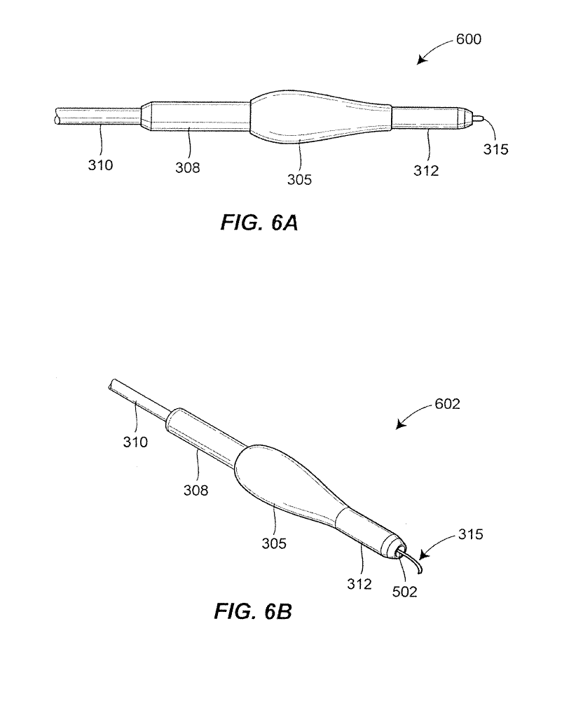

FIG. 6A illustrates a horizontally-oriented, external view 600 of the device of FIGS. 5A and 5B. In the external view 600, the adaptor 305 is securely attached to the hand-piece 308, and the insert 312 including the tip 315 is securely inserted into the hand-piece 308. FIG. 6B illustrates another external view 602 of the device of FIG. 6A. In the view 602, the insert light-exiting aperture 502 of the insert 312 is visible.

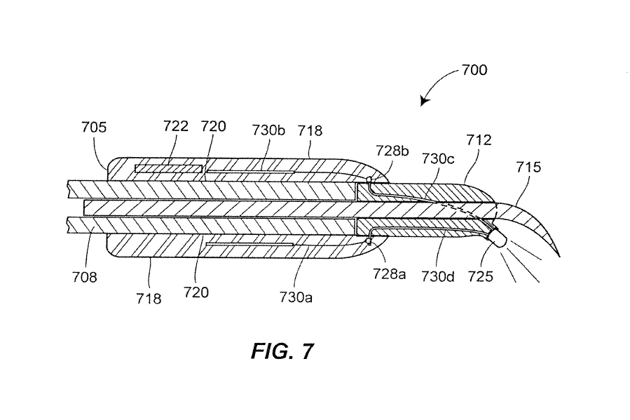

FIG. 7 illustrates an embodiment of a magnetostrictive device 700 that includes an adaptor 705 coupled to a hand-piece 708, and an insert 712 with a tip 715 securely engaged within the hand-piece 708. The magnetostrictive device 700 may be (but is not required to be) a magnetostrictive hand-held dental device and may operate in conjunction with the ultrasonic dental system 10 of FIGS. 1 and 2.

In FIG. 7, similar to FIG. 4B, the adaptor 705 may include an outer wall 718 and an inner wall 720, where the inner wall 720 encircles a housing of the hand-piece 708. In the embodiment 700 of FIG. 7, however, the adaptor 705 may include an electric power storage device 722 disposed between its outer 718 and inner 720 walls. The electric power storage device 722 may be in electrical connection with a light source, and may be of a size sufficient to illuminate the light source. The electric power storage device 722 may be any known self-contained electrical storage device that generates electricity independently of a magnetic field produced by the magnetostrictive device 700. For example, the electrical storage device 722 may be a single battery or may be a battery pack. The electrical storage device 722 may be any suitable type of known disposable, replaceable or rechargeable battery. In some embodiments, the electric power storage device or power pack 722 may be another type of power density device, such as a supercapacitor, an electrochemical double layer capacitor (EDLC), or an ultracapacitor. Other embodiments of the electrical storage device 722 may be possible.

In the embodiment 700 of FIG. 7, a light source is depicted as an LED 725 external to the insert 712 and disposed near the tip 715. The light source 725 receives power from the electrical storage device 722 via one or more electrical connections 728a, 728b that may be established when the insert 712 is engaged within the hand-piece 708 and the adaptor 705 is securely connected to the hand-piece 708. Both the adaptor 705 and the insert 712 may include wires or other electrical connections (for example, references 730a, 730b, 730c, 730d) that may enable a completion of the electric circuit between the electrical storage device 722 and the light source 725.

While the light source 725 is depicted in the embodiment 700 as being an LED disposed near the tip 715 of the insert 712, this illustrated configuration is only exemplary. In fact, the light source may be any known type of light generating source, such as one or more incandescent bulbs, neon bulbs, or light rings. Moreover, the light source need not be disposed near the tip 715 of the insert 712. In some embodiments, the light source may be disposed within the housing of the insert 712, and the insert 712 may include an insert light channel configured to channel the light from the light source through the insert 712 to an insert light-exiting aperture near the tip 715, such as the insert light-exiting aperture 502 shown in FIG. 6B.

Indeed, in some embodiments, the light source may not be disposed in the insert 712 at all. For example, the light source may be disposed within the walls 718, 720 of the adaptor 705 and may be in direct electrical connection with the electric power storage device 722. In these embodiments, the adaptor 705 may further include within its walls 718, 720 an adaptor light channel such as, for example, a first light pipe. When the adaptor 705 is securely attached to the hand-piece 708, a portion of the adaptor 708 may align with a portion of the insert 712 so that the adaptor light channel is aligned with an insert light-entering aperture of an insert light channel disposed within the insert, such as the insert light-entering aperture 507 of FIG. 5B. The insert light channel may be, for example, a second light pipe, and may be configured to channel the light through the insert 712 to an insert light-exiting aperture near the 715 tip of the insert 712. In this manner, light generated by the light source may be directed through the adaptor light channel and into the insert light channel, and the light may exit the insert 712 to illuminate an area surrounding the tip 715.

In other embodiments, the light source may be disposed within the hand-piece 708, the hand-piece may include a hand-piece light channel, and the insert 712 may include an insert light channel. When the insert 712 is engaged within the hand-piece 708, the hand-piece light channel and the insert light channel may be aligned. Furthermore, when the adaptor 705 is securely attached to the hand-piece 708, a secure electrical connection may be established between the electric power storage device 722 within the adaptor 705 and the light source within the hand-piece 708. In this manner, the electric power storage device 722 may power the light source disposed within the hand-piece 708, and light generated by the light source may be directed or channeled through the hand-piece light channel into the insert light channel and through the insert light channel to exit the insert 712 to illuminate the area surrounding the tip 715.

In yet other embodiments, the light source may be disposed within the hand-piece 708, the hand-piece 708 may include a hand-piece light channel, the insert 712 may include an insert light channel, and the adaptor 705 may include an adaptor light channel within its walls 718, 720. When the adaptor 705 is securely attached to the hand-piece 708, the adaptor 705 may be aligned with the hand-piece and the insert so that the hand-piece light channel, the adaptor light channel and the insert light channel are sequentially aligned. Furthermore, a secure electrical connection may be established between the electric power storage device 722 within the adaptor 705 and the light source 708 within the hand-piece 708. In this manner, the electric power storage device 722 may provide power to the light source 708, and the light generated by the light source may be directed or channeled through the hand-piece light channel into the adaptor light channel, through the adaptor light channel into the insert light channel, and through the insert light channel to exit the insert 712 to illuminate the area surrounding the tip 715.

As discussed above, embodiments of the present disclosure may operate in conjunction with the light source being located within the hand-piece 708, the adaptor 705 or the insert 712. Irrespective of the location of the light source, however, when the adaptor 708 is in full, secure connection with the hand-piece 708 and the insert 712 is fully engaged within the hand-piece, at least some portion of the adaptor 708 may abut at least some portion of the insert 712. An electrical connection to the light source may be established and light channels within the adaptor 705, hand-piece 708 and/or insert 712 (as appropriate) may be aligned so that light from the light source may be delivered to illuminate an area proximate to the tip 715. As power to the light source is generated by the electric power storage device 722 and thus is generated independently of a magnetic field produced by the magnetostrictive device, the intensity of the generated light will not vary based on power fluctuations at the magnetostrictive device.

A user-activated switch (not shown) may be provided so that a user of the device 700 may control whether or not the light 725 is on or off while the tip 715 is vibrating. For example, when the user desires the light 725 to turn off, he or she may indicate “off” via the user-activated switch. The user-activated switch may interrupt one or more of the electrical connections 728a, 728b, 730a-730d between the electrical storage device 722 and the light source 725. Similarly, when the user desires the light 725 to turn on, he or she may indicate “on” via the user-activated switch, and the user-activated switch may re-establish the one or more electrical connections 728a, 728b, 730a-730d. The user-activated switch may be physically located on the exterior of the hand-piece 708, the adaptor 705 or the insert 712, or the user-activated switch may be remotely located, for example, on a separate hand- or foot-operated control in communicative connection with the device 700. In any event, the user-activated switch may preferably be sealed or otherwise configured to prevent water, saliva, debris or other undesired artifacts from entering the hand-piece 708, the adaptor 705 and/or the insert 302. Other embodiments of a user-activated switch may alternatively or additionally be possible.

For embodiments where the electrical storage device 722 is a rechargeable, self-contained battery or electrical storage device, the battery may be recharged, for example, by removing the battery 722 from the adaptor 705, seating the battery into a separate charging device, allowing the separate charging device to charge the battery, and returning the battery to the adaptor 705 after it has been charged. In another example, the electrical storage device 722 may be a battery pack with a charging connection port, and the battery pack may be recharged by plugging a charging cord directly into the charging connection port.

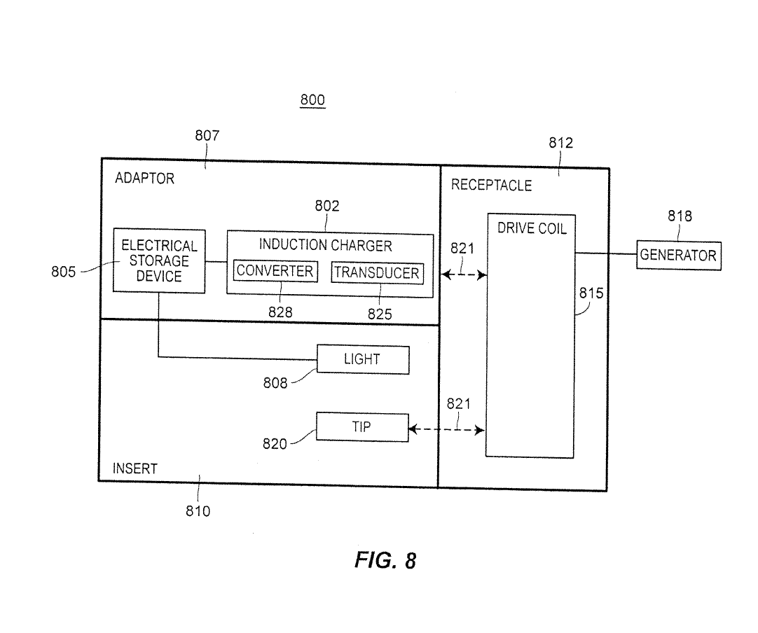

In yet another example, the electrical storage device 722 may be partially or entirely recharged via induction. FIG. 8 illustrates an embodiment, in a block diagram form, of a magnetostrictive ultrasonic device 800 that includes a charging device or induction charger 802 for a rechargeable electric power storage device or power pack 805 disposed within an adaptor 807. The rechargeable electric power storage device 805 may power a light source 808 of the device 800. The embodiment illustrated in FIG. 8 shows the light source 808 as being disposed within an insert 810, but as previously discussed, in other embodiments, the light source 808 may be disposed within the adaptor 807 or within a receptacle/hand-piece 812 into which the insert 810 may be engaged. The induction charger 802 may operate in conjunction with embodiments of the dental system 10 of FIGS. 1 and 2, and/or with embodiments of the magnetostrictive device 700 of FIG. 7.

Similar to FIGS. 1, 2 and 7, the magnetostrictive device 800 may have an insert 810 coupled to a hand-piece or other type of receptacle 812. The hand-piece or receptacle 812 may include a drive coil circuit 815 that receives alternating current from a generator or source 818 and induces an alternating magnetic field 821. The insert 810 may include a tip 820 that may vibrate or move in response to the alternating magnetic field 821. The adaptor 807 may be securely attached to the hand-piece 812, and at least a portion of the adaptor 807 may abut at least a portion of the insert 810.

The magnetostrictive ultrasonic device 800 may also include an induction charger or charging device 802 for recharging all or part of the electrical storage device 805. The induction charger or charging device 802 may be electrically coupled to the electrical storage device 805 and may be inductively coupled to the drive coil 815. For example, the induction charger 802 may include a transducer 825 that is positioned to be inductively coupled to the drive coil 815. The transducer 825 may produce a current that is induced by the magnetic field 821 that is produced when a current is applied to the drive coil 815. In some embodiments, the transducer 825 may be an induction coil, but other types of suitable transducers 825 may be used in conjunction with the present disclosure.

The induced current produced by the transducer 825 may be converted or modified 828 into a current format that may flow to the rechargeable electrical storage device 805 and may effect a re-charging of some or all of the electrical storage device 805. For example, the current from the transducer 825 may be inverted, rectified, or otherwise converted in a manner known in the art. Thus, generally speaking, the magnetic field 821 induced by the drive coil 815 that causes the tip 820 of the insert 510 to vibrate may also be used to inductively recharge the electrical storage device 805 that supplies power to the light source 808 of the device 800.

In some embodiments, the electrical storage device 805 may be re-charged by physically coupling the adaptor 807 not to the hand-piece or receptacle 812 but to a re-charging or docking station (not shown). The re-charging or docking station may be a separate physical entity from the adaptor 807, the insert 810, and the receptacle 812. The re-charging or docking station may be configured to securely receive the adaptor 807 (e.g., the adaptor 807 may be seated into the docking station) to enable a re-charging the electrical storage device 805, and upon a partial or complete re-charging of the electrical storage device 805, the adaptor 807 may be uncoupled from the re-charging or docking station. The re-charging station may include a magnetic field source such as a coil to which electric current is applied, a permanent magnet or other magnetic field source. In some embodiments, the re-charging station may include an electrical connection to an electric power source, such as when the re-charging station includes the coil to which electric current is applied. When the adaptor 807 is physically coupled to the re-charging station, the transducer 825 of the induction charger 802 may produce a current in response to the magnetic field provided by the re-charging station, and the current may effect a total or partial re-charging of the electrical storage device 805.

While FIG. 8 illustrates the induction charger 802 as included in the adaptor 807, in other embodiments, the induction charger 802 may be omitted from the adaptor 807 but included in the hand-piece or receptacle 812. Alternatively, the induction charger 802 may be a separate physical entity altogether from the adaptor 807, the insert 810 and the hand-piece or receptacle 812. For example, the induction charger 802 may reside in the re-charging or docking station along with the magnetic field source. In this example, when the electrical storage device 805 of an adaptor 807 needs re-charging, the adaptor 807 may be physically coupled to the re-charging station so that an electrical connection between the electrical storage device 805 and the induction charger 802 within the re-charging station may be established.

Variations on initiating the inductive recharging of the electrical storage device 805 may be possible. For example, in some embodiments, recharging may only occur when an electrical storage device 805 has drained down to a certain level. In some embodiments, recharging may initiate in response to a user indication. Other variations of inductively recharging the electrical storage device 805 may be contemplated and operate in conjunction with the contents of the present disclosure.

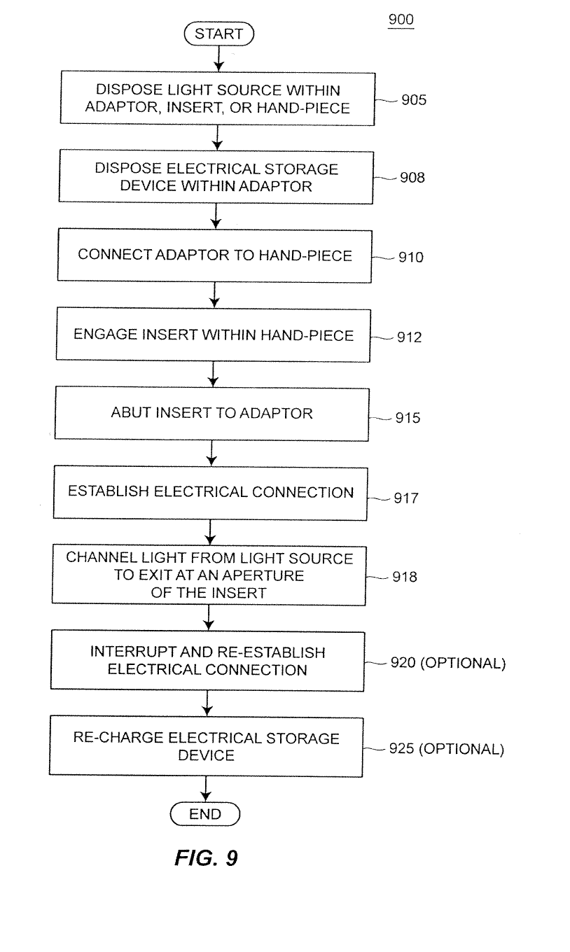

FIG. 9 illustrates an exemplary embodiment of a method 900 of providing light at a magnetostrictive hand-held device. The method 900 may be used in conjunction with any or all embodiments of the present disclosure described with respect to FIGS. 1, 2, 7 and 8.

At the start, the method 900 may include disposing 905 a light source within an adaptor, insert or hand-piece of the magnetostrictive device. The light source may be any type of electrically generated light source, such as an LED, an incandescent light, a fluorescent light, a light ring, a neon light, or other type of light source.

At block 908, an electrical storage device may be disposed within the adaptor. The electrical storage device may be a single battery, a battery pack, an electric double-layer capacitor, or other known electrical storage device. The electric power storage device may be replaceable or rechargeable. The electric power storage device may be self-contained, and may be configured to generate electric power independently of a magnetic field generated by the magnetostrictive hand-held device.

At the block 910, the adaptor may be securely connected to the hand-piece. For example, the adaptor may be slid over the hand-piece into a secure position, the adaptor may be latched onto the hand-piece, or the adaptor may be connected by some other secure means. At block 912, the insert may be securely engaged within the hand-piece of the magnetostrictive device.

At block 915, at least in part by virtue of the connections performed at the blocks 910 and 912, at least a portion of the insert may abut to the at least a portion of the adaptor. At block 917, at least in part by virtue of the blocks 910, 912 and 915, an electrical connection between the electrical storage device and the light source may be established. Additionally, at least in part by virtue of the blocks 910, 912 and 915, a light channel path from the light source through the insert may be established.

At block 918, light may be channeled from the light source and may exit the insert at an aperture proximate to the tip of the device (or at any desired exit aperture of the insert).

In some embodiments of the method 900, an optional block 920 may include interrupting and re-establishing the electrical connection between the electrical power source and the light source based on a user input. In some embodiments, the method 900 may include dimming and/or brightening an intensity of the light via the same or a different user input. The user may thus be able to control turning the light on and off and/or adjusting the intensity of the light irrespective of whether or not the tip is vibrating.

In some embodiments of the method 900, an optional block 925 may include re-charging at least a portion of the electrical storage device. For example, re-charging at least a portion of the electrical storage device may include seating the electrical storage device or the adaptor into a docking station such as described with respect to FIG. 8.

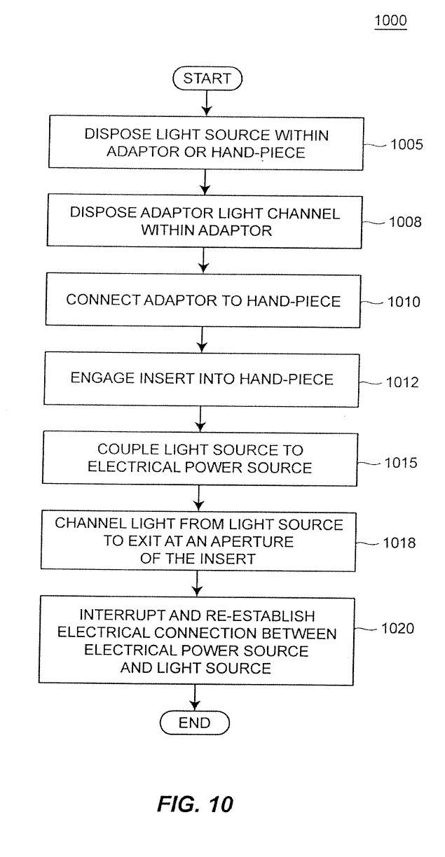

FIG. 10 illustrates an exemplary embodiment of a method 1000 of providing light at a magnetostrictive hand-held device. The method 1000 may be used in conjunction with any or all embodiments of the present disclosure described with respect to FIGS. 1, 2, and 3A-6B.

At the start, the method 1000 may include disposing 1005 a light source within an adaptor or within a hand-piece of the magnetostrictive device. The light source may be any type of electrically generated light source, such as an LED, an incandescent light, a fluorescent light, alight ring, a neon light, or other type of light source.

At block 1008, an adaptor light channel may be disposed within the adaptor. The adaptor light channel may be a light pipe disposed longitudinally along one or more sides of the adaptor, or the adaptor light channel may encircle an inner air core of the adaptor along its length. Other configurations of the adaptor light channel may be possible.

At the block 1010, the adaptor may be securely connected to the hand-piece. For example, the adaptor may be slid over and secured to the hand-piece, the adaptor may be latched onto the hand-piece, or the adaptor may be connected by some other secure means. At block 1012, the insert may be securely engaged within the hand-piece of the magnetostrictive device. At least in part by virtue of the blocks 1010 and 1012, at least a portion of the insert may abut at least a portion of the adaptor so that the adaptor light channel and an insert light channel may be aligned. If the light source is disposed within the hand-piece, a hand-piece light channel and the adaptor light channel may be aligned so that light may be delivered or channeled from the hand-piece into the adaptor.

At block 1015, the light source may be coupled to an electric power source. The electric power source may be a generator or may be a standard electrical wall outlet. The electric power source may be self-contained, and may be a single battery, a battery pack, an electric double-layer capacitor, or other known electrical storage device. The electric power source may be replaceable or rechargeable. The electric power source may be configured to generate electric power independently of a magnetic field generated by the magnetostrictive hand-held device.

At block 1018, at least in part by virtue of the blocks 1010 and 1012, light may be channeled from the light source, through the adaptor light channel, and to the insert. The light may exit the insert at an aperture proximate to the tip of the device (or at any desired exit aperture of the insert).

In some embodiments of the method 1000, an optional block 1020 may include interrupting and re-establishing the electrical connection between the electric power source and the light source based on a user input. The method 1000 may include dimming and/or brightening a generated intensity of the light source based on the same or a different user input. The user may thus be able to control turning the light on and off and/or its intensity.

It should also be understood that, unless a term is expressly defined in this patent using the sentence “As used herein, the term ‘_’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

Although the forgoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

What is claimed is:

1. A lighting system for a hand-held dental delivery device, comprising:
a hand-piece of the hand-held dental delivery device, the hand-piece including a coil, wherein an alternating current applied to the coil induces an alternating magnetic field;
an insert insertably coupled to the hand-piece, the insert including:
a tip configured for use in a dental procedure and coupled to a magnetic element responsive to the alternating magnetic field, the magnetic element being one of a stack of metal plates or at least one rod, and the magnetic element disposed within the hand-piece;
an insert light-exiting aperture proximate to the tip through which light generated by a light source is outwardly directed from the insert; and
a insert light channel disposed within the insert, the insert light channel including a first end in connection with the insert light-exiting aperture and a second end; and
an adaptor removably coupled to the hand-piece, the adaptor including a self-contained electrical storage device in electrical connection with the light source, wherein:
the light source is disposed within one of the hand-piece, the adaptor or the insert, and
the self-contained electrical storage device is configured to generate power independently of the alternating magnetic field.

2. The lighting system of claim 1, wherein:
the light source is disposed within the adaptor; and
the adaptor further includes an adaptor light channel configured to direct the light generated by the light source into an insert light-entering aperture of the insert in connection with the second end of the insert light channel.

3. The lighting system of claim 1, wherein the light source is disposed within the hand-piece, and one of:
the adaptor further includes an adaptor light channel configured to direct the light generated by the light source into an insert light-entering aperture of the insert in connection with the second end of the insert light channel; or
the lighting system further comprises a hand-piece light channel configured to direct the light generated by the light source into the insert light-entering aperture of the insert in connection with the second end of the insert light channel.

4. The lighting system of claim 1, wherein the light source is disposed within the insert so that the light generated by the light source is received by the second end of the insert light channel.

5. The lighting system of claim 1, further comprising a user-activated switch to interrupt and re-establish the electrical connection between the light source and the self-contained electrical storage device.

6. The lighting system of claim 1, wherein the self-contained electrical storage device is rechargeable.

7. The lighting system of claim 6, wherein:
the lighting system further comprises a charging device configured to electrically couple to the self-contained electrical storage device and to inductively couple to the coil, and
the alternating magnetic field induces the charging device to charge the self-contained electrical storage device.

8. The lighting system of claim 7, wherein the charging device comprises a docking station and the docking station
is configured to removably receive the adaptor;
is coupled to an electric power source, and
is physically separate from the hand-piece, the insert and the adaptor.

9. The lighting system of claim 7, wherein the charging device is disposed within the adaptor.

10. The lighting system of claim 1, wherein the adaptor has an annular shape and is configured to securely receive at least a portion of the hand-piece.

11. The lighting system of claim 1, wherein at least a portion of the adaptor is configured to securely abut at least a portion of the insert.

12. A lighting system for a hand-held dental delivery device, comprising:
a hand-piece of the hand-held dental delivery device, the hand-piece including a coil, wherein an alternating current applied to the coil induces an alternating magnetic field;
an insert insertably coupled to the hand-piece, the insert including:
a tip configured for use in a dental procedure and coupled to a magnetic element responsive to the alternating magnetic field, the magnetic element being one of a stack of metal plates or a rod, and the magnetic element disposed within the hand-piece;
an insert light-exiting aperture proximate to the tip through which light generated by a light source is outwardly directed from the insert; and
a insert light channel disposed within the insert, the insert light channel including a first end in connection with the insert light-exiting aperture and a second end; and
an adaptor removably coupled to the hand-piece, the adaptor including an adaptor light channel configured to direct the light generated by the light source into an insert light-entering aperture of the insert in connection with the second end of the insert light channel, wherein:
the light source is disposed within one of the hand-piece or the adaptor and is electrically coupled to an electric power source, and
the electric power source is configured to provide power independently of the alternating magnetic field.

13. The lighting system of claim 12, wherein:
the light source is disposed within the adaptor; and
the electric power source comprises a self-contained electrical storage device disposed within the adaptor.

14. The lighting system of claim 12, wherein:
the light source is disposed within the adaptor; and
the electric power source comprises an external electric power source externally disposed to the hand-piece, the insert and the adaptor.

15. The lighting system of claim 12, wherein the electric power source comprises a same electric power source as an electric power source delivering power to the hand-held dental delivery device.

16. The lighting system of claim 12, wherein:
the light source is disposed within the hand-piece, and
the light generated by the light source is directed from the hand-piece into the adaptor light channel.

17. The lighting system of claim 12, further comprising a user-activated switch to interrupt and re-establish an electrical connection between the light source and the electric power source.

18. The lighting system of claim 12, wherein the adaptor has an annular shape and is configured to securely receive at least a portion of the hand-piece.

19. The lighting system of claim 12, wherein at least a portion of the adaptor is configured to securely abut at least a portion of the insert.

20. A method of providing light at a magnetostrictive hand-held device, comprising:
disposing a light source within an adaptor, an insert of the magnetostrictive hand-held device, or a hand-piece of the magnetostrictive hand-held device;
disposing a self-contained electrical storage device within the adaptor, the self-contained electrical storage device configured to generate power independently of a magnetic field generated by the magnetostrictive hand-held device;
insertably coupling the insert into the hand-piece of the magnetostrictive hand-held device, and securely and removably attaching the adaptor to the hand-piece so that the light source is in electrical connection with the self-contained electrical storage device, wherein the insert includes a tip and an insert light-exiting aperture disposed proximate to the tip;
abutting at least a portion of the insert to at least a portion of the adaptor; and
channeling the light generated by the light source through the insert to exit the insert via the insert light-exiting aperture.

21. The method of claim 20, wherein the light source is disposed within the adaptor, and the method further comprises:
disposing an adaptor light channel within the adaptor; and
directing the light generated by the light source
through the adaptor light channel to exit the adaptor via an adaptor light-exiting aperture,
through the adaptor light-exiting aperture into an insert light channel disposed within the insert, and
through the insert light channel to the insert light-exiting aperture.

22. The method of claim 20, wherein the light source is disposed within the hand-piece and the method further comprises:
disposing a hand-piece light channel within the hand-piece;
directing the light generated by the light source through the hand-piece light channel into an insert light channel disposed within the insert; and
directing the light through the insert light channel to the insert light-exiting aperture.

23. The method of claim 20, wherein the light source is disposed within the hand-piece and the method further comprises:
disposing an adaptor light channel within the adaptor; and
directing the light generated by the light source
from the hand-piece into the adaptor light channel,
through the adaptor light channel to exit the adaptor via an adaptor light-exiting aperture,
through the adaptor light-exiting aperture into an insert light channel disposed within the insert, and
through the insert light channel to the insert light-exiting aperture.

24. The method of claim 20, wherein the light source is disposed within the insert, and the method further comprises directing the light generated by the light source through an insert light channel disposed within the insert to the insert light-exiting aperture.

25. The method of claim 20, wherein the self-contained electrical storage device is a rechargeable self-contained electrical storage device, the magnetic field is an alternating magnetic field produced by a current applied to a coil of the magnetostrictive hand-held device, and the method further comprises:
coupling the rechargeable self-contained electrical storage device to a charging device; and
inductively coupling the charging device to the coil so that the alternating magnetic field induces the charging device to charge at least a portion of the rechargeable self-contained electrical storage device.

26. The method of claim 25, further comprising one of:
disposing the charging device in a docking station, wherein
the docking station includes a connection to an electric power source, is uncoupled from the hand-piece, the insert and the adaptor, and is configured to receive the adaptor; and
coupling the rechargeable self-contained electrical storage device to the charging device comprises seating the adaptor into the docking station; or
disposing the charging device within one of the adaptor or the hand-piece, wherein coupling the rechargeable self-contained electrical storage device to the charging device comprises electrically coupling the rechargeable self-contained electrical storage device and the charging device.

27. The method of claim 20, further comprising interrupting and re-establishing the electrical connection between the light source and the self-contained electrical storage device via a user-activated switch.

28. The method of claim 20, wherein the magnetostrictive hand-held device is a magnetostrictive hand-held dental device.

29. A method of providing light at a magnetostrictive hand-held device, comprising:
disposing a light source within an adaptor or a hand-piece of the magnetostrictive hand-held device;
disposing an adaptor light channel within the adaptor, the adaptor light channel configured to direct light generated by the light source into an insert configured to be coupled to the magnetostrictive hand-held device;
insertably coupling the insert into the hand-piece of the magnetostrictive hand-held device, and securely and removably attaching the adaptor to the hand-piece so that light generated by the light source is directed through the adaptor light channel into the insert, wherein the insert includes a tip and an insert light-exiting aperture disposed proximate to the tip;
coupling the light source to an electric power source, the electric power source configured to provide electric power independently of a magnetic field generated by the magnetostrictive hand-held device; and
channeling the light generated by the light source through the adaptor light channel and through the insert to exit the insert via the insert light-exiting aperture.

30. The method of claim 29, wherein the light source is disposed within the adaptor, the electric power source is a self-contained electrical storage device, and the method further comprises:
disposing the self-contained electrical storage device within the adaptor; and
directing the light generated by the light source
through the adaptor light channel to exit the adaptor via an adaptor light-exiting aperture,
through the adaptor light-exiting aperture into an insert light channel disposed within the insert, and
through the insert light channel to the insert light-exiting aperture.

31. The method of claim 29, wherein:
the light source is disposed within the adaptor;
the electric power source is an external electric power source externally disposed to the hand-piece, the insert and the adaptor; and
the method further comprises directing the light generated by the light source
through the adaptor light channel to exit the adaptor via an adaptor light-exiting aperture,
through the adaptor light-exiting aperture into an insert light channel disposed within the insert, and
through the insert light channel to the insert light-exiting aperture.

32. The method of claim 29, wherein the light source is disposed within the hand-piece and the method further comprises directing the light generated by the light source
from the hand-piece into the adaptor light channel,
through the adaptor light channel to exit the adaptor via an adaptor light-exiting aperture,
through the adaptor light-exiting aperture into an insert light channel disposed within the insert, and
through the insert light channel to the insert light-exiting aperture.

33. The method of claim 29, wherein securely attaching the adaptor to the hand-piece comprises sliding the adaptor into a secure position on the hand-piece.

34. The method of claim 29, further comprising interrupting and re-establishing an electrical connection between the light source and the electric power source via a user-activated switch.

35. The method of claim 29, wherein the magnetostrictive hand-held device is a magnetostrictive hand-held dental device.

36. An adaptor for use with a lighted magnetostrictive device, comprising:
a housing of the adaptor; and
a self-contained electrical storage device disposed within the housing of the adaptor, in electrical connection with a light source, and configured to generate electric power independently of a magnetic field generated by the lighted magnetostrictive device,
wherein:
the adaptor is configured to be securely and removably attached to a hand-piece of the lighted magnetostrictive device,
at least a portion of the adaptor is configured to securely abut at least a portion of an insert, the insert being securely and insertably attached to the hand-piece of the lighted magnetostrictive device, and
the light source is disposed within the housing of the adaptor, a housing of the hand-piece, or a housing of the insert.

37. The adaptor of claim 36, wherein:
the light source is disposed within the housing of the adaptor;
the adaptor further includes an adaptor light channel; and
light generated by the light source is channeled
through the adaptor light channel to an adaptor light-exiting aperture,
through the adaptor light-exiting aperture into an insert light channel disposed within the insert, and
through the insert light channel to exit the insert via an insert light-exiting aperture disposed at an end of the insert distal to the hand-piece.

38. The adaptor of claim 36, wherein:
the light source is disposed within the housing of the hand-piece;
the adaptor further includes an adaptor light channel; and
light generated by the light source is channeled
from the hand-piece into the adaptor light channel,
through the adaptor light channel to an adaptor light-exiting aperture,
through the adaptor light-exiting aperture into an insert light channel disposed within the insert, and
through the insert light channel to exit the insert via an insert light-exiting aperture disposed at an end of the insert distal to the hand-piece.

39. The adaptor of claim 36, wherein:
the light source is disposed within the housing of the insert; and
light generated by the light source is directed through an insert light channel disposed within the insert to exit the insert via an insert light-exiting aperture at an end of the insert distal to the hand-piece.

40. The adaptor of claim 36, wherein:
the lighted magnetostrictive device comprises a lighted magnetostrictive dental device including a tip enabled to move in response to the magnetic field,
the magnetic field is an alternating magnetic field generated by current applied to a coil disposed within the hand-piece, and
light generated by the light source exits the insert to illuminate at least a portion of an area proximate to the tip.

41. The adaptor of claim 36, wherein the self-contained electrical storage device comprises a rechargeable self-contained electrical storage device.

42. The adaptor of claim 41, wherein:
the rechargeable self-contained electrical storage device is configured to electrically couple to a charging device,
the charging device is configured to inductively couple to a coil disposed within the hand-piece, and
the magnetic field is generated by current applied to the coil and induces the charging device to charge the rechargeable self-contained electrical storage device.

43. The adaptor of claim 42, wherein:
the charging device is configured to removably receive the adaptor, includes a connection to an electric power source, and is externally disposed to and physically separate from the housing of the adaptor, the housing of the hand-piece and the housing of the insert; or
the charging device is disposed within the housing of the adaptor.

44. The adaptor of claim 36, wherein the adaptor has an annular shape and is configured to securely receive at least a portion of the hand-piece.

45. The adaptor of claim 36, further comprising a switch for interrupting and re-establishing the electrical connection between the light source and the self-contained electrical storage device.

46. An adaptor for use with a lighted magnetostrictive device, comprising:
a housing of the adaptor; and
an adaptor light channel disposed within the housing and configured to direct light generated by a light source to an adaptor light-exiting aperture,
wherein:
the adaptor is configured to be securely and removably attached to a hand-piece of the lighted magnetostrictive device,
at least a portion of the adaptor is configured to securely abut to at least a portion of an insert, the insert being securely and insertably attached to the hand-piece of the lighted magnetostrictive device,
the light source is disposed within one of the housing of the adaptor or a housing of the hand-piece, and is electrically connected to an electric power source, and
the electric power source is configured to provide electric power independently of a magnetic field generated by the lighted magnetostrictive device.

47. The adaptor of claim 46, wherein:
the light source is disposed within the housing of the adaptor;
the electric power source comprises one of:
a self-contained electrical storage device disposed within the housing of the adaptor, or
an external electric power source externally disposed to the hand-piece, the insert and the adaptor; and
the light generated by the light source is channeled:
through the adaptor light channel to the adaptor light-exiting aperture,
through the adaptor light-exiting aperture into an insert light channel disposed within the insert, and
through the insert light channel to exit the insert via an insert light-exiting aperture disposed at an end of the insert distal to the hand-piece.

48. The adaptor of claim 46, wherein:
the light source is disposed within the housing of the hand-piece;
the electric power source comprises one of:
a self-contained electrical storage device disposed within the housing of the adaptor, or
an external electric power source externally disposed to the hand-piece, the insert and the adaptor; and
the light generated by the light source is channeled:
from the hand-piece into the adaptor light channel,
through the adaptor light channel to the adaptor light-exiting aperture,
through the adaptor light-exiting aperture into an insert light channel disposed within the insert, and
through the insert light channel to exit the insert via an insert light-exiting aperture disposed at an end of the insert distal to the hand-piece.

49. The adaptor of claim 46, wherein:
the lighted magnetostrictive device comprises a lighted magnetostrictive dental device including a tip enabled to move in response to the magnetic field,
the magnetic field comprises an alternating magnetic field generated by a current applied to a coil disposed within the hand-piece, and
the light generated by the light source exits the insert to illuminate at least a portion of an area proximate to the tip.

50. The adaptor of claim 46, wherein the adaptor has an annular shape and is configured to securely receive at least a portion of the hand-piece.
